(19) United States
(12) Patent Application Publication
(10) Pub. No.: US 2020/0200740 A1
(43) Pub. Date: Jun. 25, 2020

(54) METHOD FOR DETECTING EXTRACELLULAR VESICLES IN A SAMPLE

(71) Applicant: FORSCHUNGSZENTRUM JUELICH GMBH, JUELICH (DE)

(72) Inventors: Christian ZAFIU, Vienna (AU); Dieter Willbold, Juelich (DE); Andreas Kulawik, Erkrath (DE); Oliver Bannach, Duesseldorf (DE)

(21) Appl. No.: 16/612,773

(22) PCT Filed: May 16, 2018
(86) PCT No.: PCT/DE2018/000145

§ 371 Date: Nov. 12, 2019

(30) Foreign Application Priority Data
Jun. 13, 2017 (DE) 10 2017 005 543.2

(51) Int. Cl. G01N 33/53; G01N 21/64; G01N 33/543; G01N 1/34; G01N 33/552

(52) U.S. Cl. CPC G01N 33/5308; G01N 21/6458; G01N 33/552; G01N 1/34; G01N 33/543

(57) ABSTRACT

A method for detecting extracellular vesicles in a sample, including the following steps: (a) applying the sample to a substrate, (b) adding probes suitable for detection which mark the extracellular vesicles by specific binding to them; and (c) detecting the extracellular vesicles by measuring a specific signal from the probes; wherein step (b) can be performed prior to step (a).

### CROSS-REFERENCE TO PRIOR APPLICATIONS

This application is a U.S. National Phase application under 35 U.S.C. § 371 of International Application No. PCT/DE2018/000145, filed on May 16, 2018, and claims benefit to German Patent Application No. 10 2017 005 543.2, filed on Jun. 13, 2017. The International Application was published in German on Dec. 20, 2018 as WO 2018/228625 A1 under PCT Article 21(2).

### FIELD

Method for detecting extracellular vesicles in a sample

The invention relates to a method for detecting extracellular vesicles in a sample.

### BACKGROUND

Extracellular vesicles (EVs) are membrane particles that can be secreted by almost any cell and reabsorbed by a variety of cells. These vesicles can transfer information from one cell to other cells. Three classes of extracellular vesicles are distinguished: Exosomes, smaller than 100 nm in diameter, originating from endosomes inside the cell. Larger microparticles (100-1000 nm) which separate directly from the cell membrane. The third class of extracellular vesicles are vesicles which form during apoptosis. The vesicles may be generated by various factors, such as extracellular stimuli, microbial infections, and other stress factors. Extracellular vesicles consist of a lipid bilayer in which membrane proteins are integrated and a solution inside.

Inside the extracellular vesicles there may be proteins, DNA and/or RNA, which are called cargo. A few proteins are found in all extracellular vesicles and are secreted independent of cell type [1]. These include proteins found inside cells, such as actin and tubulin, but also membrane proteins such as the tetraspanins CD9, CD63 and CD81 as well as the class I histocompatibility complex (MHC I) [1, 2]. Other proteins, DNA and/or RNA fragments are found in certain cell types. [3].

The biological role of extracellular vesicles is complex and not yet completely clarified. It is currently known that extracellular vesicles, among other things, reject unwanted proteins from cells, but may also play a role in cell-cell communication, such as in the stimulation of the immune system. [1]. These vesicles also play a fundamental role in some life-threatening diseases such as cardiovascular, kidney and many cancers and can give a direct indication of a specific disease [3-6]. Since extracellular vesicles are released by cells into the surrounding medium and also excreted renally, they can be found in body fluids such as blood, cerebrospinal fluid, as well as in urine, where they are detected and can provide information about a disease even without a biopsy [3, 7].

Another field of application is the use of extracellular vesicles secreted by cancer cells. These could be used as vaccines [8]. This makes it possible to immunize risk patients on the one hand and on the other hand to produce biopharmaceutical active substances, for example antibodies, and to use them for therapeutic purposes. The direct use of exosomes for therapeutic purposes is also considered. [7].

The gold standard in the detection and characterization of extracellular vesicles are electron microscopic techniques, of which cryo transmission electron microscopy is the most sensitive technique. This method provides the highest resolution and the most accurate statements on the size distribution of extracellular vesicles and it is also possible to characterize extracellular vesicles with immunostaining. The disadvantage is that the sample preparation is difficult, the measurement extremely time-consuming and the method very expensive overall. It also requires well trained personnel, which means that it is neither suitable for absolute quantification nor for routine diagnostics. [9].

The most widely used method for the analysis of extracellular vesicles is the flow cytometer (FCM), which detects and counts individual vesicles and cells from mostly diluted samples on the basis of an optical property as they flow past a detector. In its standard design, the flow cytometer sorts and counts the refractive index via the light scattering. Although the scattering intensity may also give information about the size of individual particles, the lower detection limit is disadvantageously about 400 nm. In an improved version, extracellular vesicles are additionally marked with a fluorescence-marked antibody and, in addition to light scattering, the fluorescence signal is also evaluated. The advantage is that extracellular vesicles can be characterized and the size resolution limit is reduced to about 100 nm. In the most recent flow cytometers, the fluorescence signal is recorded with a camera, and different wavelengths can be excited, thus affording the possibility of detecting different types of extracellular vesicles. However, the disadvantages of the technique lie in the fact that only one property can be assigned to a certain vesicle in the flow, so that the method also detects residues from the medium if these have not been removed with great effort. The lower resolution limit is also at 100 nm for image-based flow cytometers. [9].

In the Resistive Pulse Methods (RPS), in combination with FCM, the sample solution passes through a pore between two electrodes onto which a voltage is applied. Passing particles increase the electrical resistance between the electrodes, allowing particles to be counted. The achievable lower detection limit of such methods is 40 nm and depends on the hole diameter. The disadvantage is that the pore can become clogged and that it can be difficult to find the optimal settings to count all particles in samples with heterogeneous size distributions. Moreover, the method cannot be used to characterize extracellular vesicles. [10].

Dynamic light scattering (DLS) is also used. Although it is easy to operate and has a lower detection limit of up to 5-10 nm, the method is not suitable for quantifying extracellular vesicles. Also, artifacts may interfere [9].

In nanoparticle tracking analysis (NTA), the particle movements are tracked via light scattering and recorded with a camera. Conclusions about the size distribution and concentration thereof can be obtained from the data obtained. Some devices are equipped with a fluorescence detection system which also allows the tracing of marked extracellular vesicles. For heterogeneous size distributions, however, samples must be measured in different dilutions. The lower detection limit is 50 nm [9].

Some of the state of the art methods are not able to cover the entire or the crucial (30-100 nm) size range for exosomes, underestimating the actual number of extracellular vesicles and losing important information.

The decisive disadvantage of the methods according to the prior art lies in the fact that the samples have to be purified in a complicated manner in order to be able to carry out a detection. With the methods, it is also not possible to clearly distinguish whether the measured particles are exosomes, microparticles or microvesicles or other vesicles or particles from the sample matrix. [11].

### SUMMARY

A method for detecting extracellular vesicles in a sample, including the following steps: (a) applying the sample to a substrate, (b) adding probes suitable for detection which mark the extracellular vesicles by specific binding to them; and (c) detecting the extracellular vesicles by measuring a specific signal from the probes; wherein step (b) can be performed prior to step (a).

### BRIEF DESCRIPTION OF THE DRAWINGS

FIG. 1 shows the results of quantitative evaluation of microscopy images after averaging of two samples (duplicate measurement; 50 images) and application of an intensity filter (of 4000 of 16384 intensity values). A) shows emissions at 705 nm (EM) and excitation at 633 nm (EX). This channel represents APC dyes. B) shows the determined pixels at Ex/Em=561/600 nm. This channel excites PE and mCherry dyes and C) for Ex/Em=488/600. This channel excites PE dyes.

### DETAILED DESCRIPTION

In an embodiment, the present invention provides a method for the detection of extracellular vesicles in any sample, e.g. body fluids such as blood plasma, serum, urine, cerebrospinal fluid but also cell culture supernatants.

In another embodiment, the invention provides a kit for carrying out the detection.

An embodiment of the present invention is a method for detecting extracellular vesicles in a sample, comprising the following steps:

a) applying the sample to a substrate,
b) adding probes suitable for detection which mark the extracellular vesicles by specific binding to them; and
c) detecting the extracellular vesicles by measuring a specific signal from the probe, wherein step b) can be performed prior to step a).

In one embodiment of the invention, the method is characterized in that before step a) an immobilization of capture molecules for the extracellular vesicles takes place on the substrate.

In a further embodiment of the invention, the extracellular vesicles are immobilized on the substrate by being brought into contact with the capture molecules and by binding to the capture molecules.

In a further embodimentof the invention, after bringing the extracellular vesicles into contact with the probes, molecules and particles that are not specifically bound are removed by washing.

In an embodiment, it is advantageously possible to select probes which bind to the extracellular vesicles, wherein the probes are, for example, also capable of emitting a specific signal only after binding.

In an embodiment, contacting the extracellular vesicles with the capture molecules and the probes can be done simultaneously.

In another embodiment, contacting of the extracellular vesicles with the probes may also occur prior to contacting the capture molecules.

In certain embodiments, the method allows advantageously a determination of the size distribution of extracellular vesicles, especially in the size range of 10-100 nm for exosomes and 100-1000 nm for microparticles, as shown below.

With certain embodiments of this method it is possible to detect extracellular vesicles in any sample and in a small number of extracellular vesicles. Therefore, an individual detection can also be carried out without having to clean the sample in a complex manner.

In addition, certain embodiments of the method can enable qualitative detection of extracellular vesicles and quantification and characterization in any sample. On the one hand, this advantageously ensures a direct and absolute quantification of the number of extracellular vesicles, and on the other hand a characterization of the size distribution of extracellular vesicles.

In certain embodiments, the characterization can also be carried out on the basis of the quantification and identification of proteins, DNA and RNA inside the vesicle and/or on the basis of membrane proteins.

In certain embodiments, the detection is carried out with simple steps directly on any samples. The term “any sample” also means buffers with different additives or culture media. Alternatively, the sample can be taken ex vivo from body fluids or be a body fluid. Samples from the environment, such as e. g. water, plant and soil samples, as well as food, can be examined directly, and the extracellular vesicles can be detected.

One embodiment of the method according to the invention is the quantitative and/or qualitative determination of extracellular vesicles which contain at least one binding site for a capture molecule and at least one binding site for a probe. This method comprises the following steps:

Method for the quantitative and/or qualitative determination of extracellular vesicles comprising at least one binding site for a capture molecule and at least one binding site for a probe comprising the following steps:

a) immobilizing capture molecules on a substrate,
b) contacting the extracellular vesicles with the capture molecules,
c) immobilizing the extracellular vesicles on the substrate by binding to capture molecules,
d) contacting the extracellular vesicles with the probes and
e) removing non-specifically bound molecules and particles, e. g. by washing,
f) binding the probes to the extracellular vesicles,

wherein the probes are capable of emitting a specific signal and steps b) and d) can occur simultaneously or d) before b).

Also, in certain embodiments, steps c) and f) can thus be carried out simultaneously in an advantageous manner.

In a further embodiment of the method according to the invention, in which the extracellular vesicles are brought into contact with the probes before they are brought into contact with the capture molecules, extracellular vesicles marked with probes are immobilized on the substrate.

Thus, in this embodiment, the probes are bound to the extracellular vesicles before the extracellular vesicles are brought into contact with the capture molecules and immobilized to the substrate.

In a further embodiment of the method according to the invention, the sample is chemically fixed, e.g. by formaldehyde, after the extracellular vesicles are brought into contact with the probes.

Optionally, the sample may additionally be admixed with DNA and RNA binding probes after or during or prior to binding of the probes to the extracellular vesicles.

In one embodiment of the invention, a detergent is used after the chemical fixation in order to render the membrane of the extracellular vesicle permeable and, e.g. during the binding of the probes to the extracellular vesicles, probes can penetrate into the interior of the extracellular vesicles.

For the purposes of the present invention, the “quantitative determination” first means the determination of the concentration of the extracellular vesicles, and thus can also mean the determination of their presence and/or absence.

Preferably, the quantitative determination also means the selective quantification of certain types of extracellular vesicles. Such quantification can be proven via the corresponding specific probes.

For the purposes of the present invention, the “qualitative determination” means characterization of the extracellular vesicles.

The extracellular vesicles are marked with one or more probes useful and/or specific for detection. The probes contain an affinity molecule which recognizes and binds to a binding site of the extracellular vesicle. In addition, the probes contain at least one detection molecule or a part of a molecule which is covalently bound to the molecule or part of a molecule with affinity to extracellular vesicles and which can be detected and measured by chemical or physical methods.

In one embodiment of the invention, the probes can have identical affinity molecules or molecule parts with different detection molecules (or parts). In another alternative, different affinity molecules or parts of molecules may be combined with different detection molecules or parts, or alternatively, different affinity molecules or parts may be combined with identical detection molecules or parts.

In certain embodiments, it is also possible to use mixtures of different probes.

The use of a plurality of different probes coupled to different detection molecules or molecule parts increases on the one hand the specificity of the signal (correlation signal), and on the other hand, this allows the identification of extracellular vesicles which differ in one or more characteristics. This allows selective quantification and characterization of the extracellular vesicles.

In one embodiment, a spatially resolved determination of the probe signal takes place, that is to say a spatially resolved detection of the signal emitted by the probe. Accordingly, in this embodiment of the invention, methods based on a non-spatially resolved signal, such as ELISA or sandwich ELISA, are excluded.

A high spatial resolution is advantageous in the detection. In one embodiment of the method according to the invention, so many data points are collected that they allow the detection of an extracellular vesicle in front of a background signal which is caused, for example, by device-specific noise, other unspecific signals or non-specifically bound probes. In this way, as many values are read out (read out values) as there are spatially resolved events, such as, for example, pixels. The spatial resolution determines each event against the respective background and thus represents an advantage over ELISA methods without a spatially resolved signal.

In one embodiment, the spatially resolved determination of the probe signal is based on total internal reflection fluorescence microscopy (tirfm) and the examination of a small volume element compared to the volume of the sample, in the range from a few femtoliters to below a femtoliter, or a volume range above the contact surface of the capture molecules with a height of 500 nm, preferably 300 nm, particularly preferred 250 nm, in particular 200 nm.

In certain embodiments of the invention, extracellular vesicles are detected which are selected from the group comprising or consisting of exosomes and/or microparticles.

In one embodiment, the material of the substrate is selected from the group comprising or consisting of plastic, silicon and silicon dioxide. In a preferred alternative, glass is used as the substrate.

In a further embodiment of the invention, the capture molecules are covalently bound to the substrate.

In an embodiment, a substrate having a hydrophilic surface is used for this purpose. In another embodiment, this is achieved by the application of a hydrophilic layer, prior to step a), to the substrate. Consequently, the capture molecules bind, in particular covalently, to the substrate or to the hydrophilic layer with which the substrate is loaded (or coated).

The hydrophilic layer is a biomolecule-repellent layer, so that the nonspecific binding of biomolecules to the substrate is advantageously minimized. The capture molecules are preferably covalently immobilized on this layer. These have affinity to a feature of the extracellular vesicles. The capture molecules may all be identical, or mixtures of different capture molecules may be present.

In an embodiment, the same molecules are used as capture molecules and probes. Preferably, the capture molecules do not comprise a detection molecule or molecule parts suitable for detection.

In one embodiment, the hydrophilic layer is selected from the group comprising or consisting of polyethylene glycol, poly lysine, preferably poly D lysine, and dextran or derivatives thereof, preferably carboxymethyl-dextran (CMD). Derivatives in the sense of the invention are compounds which differ in some substituents from the parent compounds, the substituents being inert with respect to the method according to the invention.

In one embodiment of the invention, the surface of the substrate is first hydroxylated and activated with amino groups prior to application of the hydrophilic layer. In another embodiment, this activation with amino groups is carried out by bringing the substrate into contact with APTES (3-aminopropyl-trietoxy silane) or with ethanolamine.

For preparing the substrate for the coating, one or more of the following steps are carried out:
washing a substrate of glass or glass carrier in an ultrasonic bath or plasma cleaner, alternatively incubating in 5 M NaOH for at least 3 hours,
rinsing with water and subsequently drying under nitrogen,
dipping into a solution of concentrated sulfuric acid and hydrogen peroxide in a ratio of 3:1 for the activation of the hydroxyl groups,
rinsing with water to a neutral pH, subsequently washing with ethanol and drying under a nitrogen atmosphere,
immersing in a solution of 3-aminopropyl-trietoxy silane (APTES) (1-7%), preferably in dry toluene, or a solution of ethanolamine,
rinsing with acetone or DMSO and water and drying under nitrogen atmosphere.

In an embodiment, the substrate is brought into contact with APTES in the gas phase; the pretreated substrate, if necessary, is therefore vaporized with APTES.

For coating with dextran, preferably carboxymethyl-dextran (CMD), the substrate is incubated with an aqueous solution of CMD in a concentration of 10 mg/ml or 20 mg/ml and optionally N-ethyl-N-(3-dimethylaminopropyl)carbodiimide (EDC), (200 mM) and N-hydroxysuccinimide (NHS), (50 mM) and subsequently washed.

In one embodiment, the carboxymethyl-dextran is covalently bonded to the glass surface, which was first hydroxylated and, in particular, functionalized with amino groups.

Microtiter plates, preferably with a glass base, can also be used as the substrate. Since the use of concentrated sulphuric acid is not possible when polystyrene frames are used, activation of the glass surface takes place in an analogous manner in an embodiment variant of the invention.

Capture molecules which have affinity to a feature of the extracellular vesicle to be detected are immobilized on this hydrophilic layer, preferably covalently. This feature may be a protein. The capture molecules may all be identical or be mixtures of different capture molecules.

In one embodiment of the present invention, the capture molecules, preferably antibodies, are immobilized on the substrate, optionally after activation of the CMD-coated carrier by a mixture of EDC/NHS (200 and 50 mM, respectively).

Remaining carboxylate end groups to which no capture molecules have been bound can be deactivated. Ethanolamine is used to deactivate these carboxylate end groups on the CMD spacer. Prior to the application of the samples, the substrates or carriers are optionally rinsed with buffer.

The sample to be measured is brought into contact with the substrate prepared in this way and optionally incubated. Endogenous fluids or tissue can be used as the sample to be examined. In one embodiment of the present invention, the sample is selected from cerebrospinal fluid (CSF), blood, plasma, and urine. The samples may undergo various processing steps known to those skilled in the art.

In one embodiment of the present invention, the sample is applied directly to the substrate, e.g., the uncoated substrate, optionally by covalent bonding. If necessary, binding to an activated surface of the substrate takes place.

In another embodimentof the present invention, the sample is pretreated according to one or more of the following process steps:
diluting with water or buffer,
treatment with enzymes, for example proteases, nuclease, lipases,
centrifuging,
precipitation,
competition with probes to displace any antibodies present.

Preferably, the sample is brought into contact with the substrate directly and/or without pretreatment.

Non-specifically bound substances can be removed by washing steps.

In a further step, the immobilized extracellular vesicles are marked with one or more probes which serve for further detection. As described herein, the individual steps can also be performed in a different order according to the invention.

By suitable washing steps, excess probes that are not bound to extracellular vesicles are removed.

In an alternative to the method, these excess probes are not removed. This eliminates one washing step and there is no equilibrium shift towards dissociation of the extracellular vesicle-probe complexes or compounds. Due to the spatially resolved detection, the excess probes are not recorded during the evaluation.

In one embodiment, the binding sites of the extracellular vesicles are epitopes and the capture molecules and probes are antibodies and/or antibody parts and/or fragments thereof. In a variant of the present invention, the capture molecules and the probes may be identical.

In one embodiment of the present invention, the capture molecules and probes differ. For example, different antibodies and/or antibody parts and/or fragments can be used as capture molecules and as probes. In a further embodiment of the present invention, capture molecules and probes are used which are identical to one another with the exception of the possible (dye) marking.

In a further embodiment of the present invention, different probes are used simultaneously.

In a further embodiment of the present invention, at least two or more different capture molecules and/or probes are used which contain, for example, different antibodies and optionally also carry different dye markings.

For detection purposes, the probes can be characterized in that they emit an optically detectable signal selected from the group consisting of fluorescence, bioluminescence and chemiluminescence emission and absorption.

In one embodiment, the probes are thus marked with fluorescent dyes. The dyes known to the person skilled in the art can be used as fluorescent dye. Alternatively, GFP (Green Fluorescence Protein), conjugates and/or fusion proteins thereof, and quantum dots may be used.

For quality control of the surface, e.g. to prove the uniformity of the coating with capture molecules, capture molecules marked with fluorescent dyes can be used.

For this purpose, a dye is preferably used which does not interfere with the detection of the fluorescent dye of the probe on the extracellular vesicle. This enables subsequent control of the structure and standardization of the measurement results.

The immobilized and marked extracellular vesicles are detected by imaging the surface, e.g. using laser scanning microscopy. As high a spatial resolution as possible determines a high number of pixels, as a result of which the sensitivity and the selectivity of the method can be increased, since structural features can also be imaged and analyzed. Thus, the specific signal in front of the background signal (e.g., non-specifically bound probes) increases.

Detection preferably takes place, for example, with spatially resolving fluorescence microscopy by a TIRF microscope, as well as the corresponding superresolution variants thereof, such as, e.g. STORM, dSTORM.

In one embodiment of the present invention, a laser focus such as is used in laser scanning microscopy, or an FCS (Fluorescence Correlation Spectroscopy System) is used for this purpose, as well as the corresponding superresolution variants such as STED, PALM or SIM.

In contrast to ELISA, these methods result in as many read out values as there are spatially resolved events (e.g. pixels). Depending on the number of different probes, this information is advantageously multiplied. This multiplication applies to each detection event and leads to an information gain since it discloses further properties, e.g. a second feature, via extracellular vesicles. By such a structure, the specificity of the signal can be increased for each event.

The probes can be selected such that the presence of individual features of extracellular vesicles, such as e.g. individual membrane proteins, do not affect the measurement result.

The probes can be selected such that extracellular vesicle species (phenotypes) can be determined for each individual extracellular vesicle.

Additional probes can be selected to differentiate between DNA/RNA-containing extracellular vesicles and thus provide information about the interior of the extracellular vesicles. For example, fluorophores which bind DNA/RNA, such as DAPI from Hoechst, can be used for this purpose.

For evaluation, the spatially resolved information, e.g. the fluorescence intensity, of all probes used and detected is used in order to determine, for example, the number of extracellular vesicles, their size and their characteristics.

In doing so, e.g. algorithms of background minimization and/or intensity threshold values can also be used for further evaluation and pattern recognition.

Further image analysis options include, for example, the search for local intensity maxima in order to obtain from the image information the number of extracellular vesicles detected and also to be able to determine the particle sizes.

In order to make the test results comparable with one another over distances, times and experiments, internal and/or external standards (controls) can be used.

In certain embodiments, the present invention also includes nanoparticle standards (controls), which have a defined size and preferably covalently carry the surface characteristics of the extracellular vesicles to be investigated.

The standards (controls) are preferably silica nanoparticles, but plastic nanoparticles are also possible.

Certain embodiments of the present invention also relate to a kit containing one or more of the following components:
substrate, optionally with hydrophilic surface,
capturing molecule,
probe,
substrate with capture molecule,
solutions,
standard (control),
buffer.

The compounds and/or components of the kit embodiments of the present invention may be packaged in containers, optionally with/in buffers and/or a solution.

Alternatively, some components may be packaged in the same container. Additionally or alternatively, one or more of the components could be absorbed on a solid carrier, such as a glass plate, a chip or a nylon membrane, or on the well of a microtiter plate. The substrate then comprises such a microtiter plate.

Further, the kit embodiments may include instructions for use of the kit for any of the embodiments.

In a further embodiment of the kit, the capture molecules described above are already immobilized on the substrate. In addition, the kit may contain solutions and/or buffers. To protect the coating and/or the capture molecules immobilized on it, they can be covered with a solution or a buffer.

A further embodiment of the present invention is the use of the method according to the invention for the detection of extracellular vesicles in any sample for the quantification and thus titer determination of extracellular vesicles.

Advantageously, the method can also provide evidence of a disease, such as cardiovascular, kidney and cancer diseases, the detection of an immune response. The method can be used in substance development, for the direct and absolute quantification of extracellular vesicles, target engagement, differential diagnostics, detection of protein-protein interaction and/or typing of extracellular vesicles.

Another embodiment of the present invention is the use of the inventive method for monitoring therapies with extracellular vesicles as well as for monitoring and/or checking the efficacy of active substances and/or therapies. The method can therefore be used in clinical tests, studies as well as in therapy monitoring. For this, samples are measured by the method according to certain embodiments of the invention and the results compared.

A further embodiment of the present invention is the implementation of the method in accordance with the invention to determine the efficacy of active substances against diseased cells. The results are compared with one another on the basis of the characterization of extracellular vesicles in samples. The samples are, accordingly, body fluids taken before, after, or at different times after the administration of the active substances or after the therapy has been performed. According to certain embodiments of the invention, the results are compared with a control which was not subjected to the active ingredient and/or therapy. The results are used to select active ingredients and/or therapies.

A further embodiment of the present invention is the execution of the method in accordance with the invention to determine if a person is to be included in a clinical study. For this purpose, samples are measured by the method according to the invention and the decision is made with respect to a limit value. The invention will be explained in more detail below with reference to a FIGURE and the associated measurement as an advantageous exemplary embodiment.

### EXAMPLES

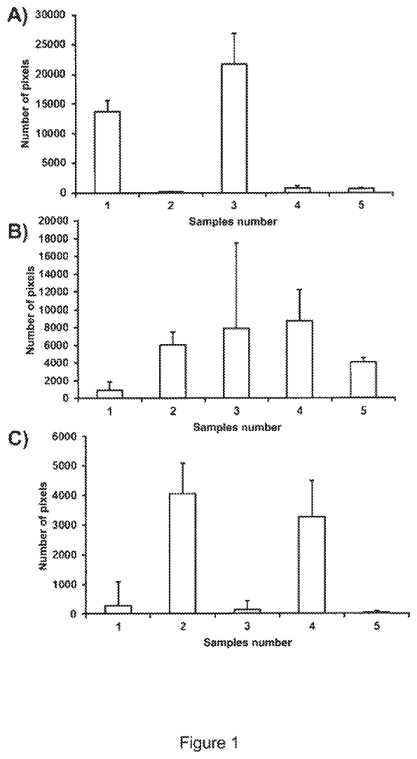

FIG. 1 shows the results of quantitative evaluation of microscopy images after averaging of two samples (duplicate measurement; 50 images) and application of an intensity filter (of 4000 of 16384 intensity values). A) shows emissions at 705 nm (EM) and excitation at 633 nm (EX). This channel represents APC dyes. B) shows the determined pixels at Ex/Em=561/600 nm. This channel excites PE and mCherry dyes and C) for Ex/Em=488/600. This channel excites PE dyes. Sample 1 are cell culture supernatants of HEK cells that do not express NEF mCherry and were treated with anti MHC1 antibodies with PE dye. Sample 2 corresponds to sample 1, with the difference that MHC1 antibodies carry an APC dye. In sample 3 there are cell culture supernatants of HEK cells expressing an NEF mCherry fusion protein and marked with anti MHC1 antibody PE. Sample 4 corresponds to sample 3, with the difference that the anti MHC1 antibody was marked with APC instead of PE. In Sample 5, cell culture supernatants from cells expressing NEF mCherry were not marked with antibodies. In A) it can be seen that sample 1 and 3 clearly differ from the other samples. These samples were treated with anti MHC1 antibodies bearing an APC dye. Thus extracellular vesicles carrying a MHC1 protein could be detected in these samples. In B), it can be seen that sample 1 has a lower number of pixels than the other samples. In this fluorescence channel PE dyes and mCherry were excited and therefore no APC. This FIGURE shows that it is possible to quantify PE (sample 2) and mCherry (sample 5), which is expressed in the cells and packaged in extracellular vesicles. The combination (Sample 3 and 4) also provides signals suitable for quantification.

C) shows the contrary image to A) and allows to quantify anti MHC1 antibodies with PE dye alone. Thus, it is also possible to measure extracellular vesicles with these antibodies.

NEF is the Negative Regulatory Factor, a protein found in exosomes. mCherry is a fluorescent protein with an absorption maximum at 558 nm and an emission maximum at 583 nm.

### Assay Structure:

Commercial microtiter plates (Greiner Bio-one; Sensoplate Plus) with 384 reaction chambers (RC) and glass bottom were used for the experiment. First, the surface of the microtiter plate was constructed. For this purpose, the plate was placed in a desiccator containing a dish containing 5% APTES in toluene. The desiccator was flooded with argon and incubated for one hour. The dish was then removed and the plate dried in vacuo for 2 hours. 20 μl of a 2 mM solution of SC-PEG-CM (MW 3400; Laysan Bio) were filled into the RC of the dry plate in de-ionized H₂O and incubated for 4 hours. After incubation, the RC was washed three times with water and then incubated with 20 μl each of an aqueous 200 mM EDC solution (1-ethyl-3-(3-dimethylaminopropyl) carbodiimide; Sigma) and with 50 mM NHS (N-hydroxysuccinimide, Sigma) for 30 minutes. The plate was again washed three times with deionized water. The RC were then coated with anti-CD63 antibodies and anti-MHCI antibodies as capture molecules (20 μl; 5 μg ml⁻¹ each antibody in PBS; 1 hour). Finally, the RC was treated with the washing program consisting in each case of three times of washing and vacuuming with TBS with 0.1% Tween-20 and TBS. In the next step, the RCs were coated with 50 μl Smartblock (Candor Bioscience GmbH) overnight at room temperature (RT) and washed again three times with saline tris-(hydroxymethyl)-aminomethane (TBS; pH=7.4). Afterwards, samples of 20 μl each were applied in triplicate in RC and incubated at RT for 1 hour. After incubation, the RCs were washed three times with TBS and loaded with 20 μl detection antibodies. Anti-MHC1 antibodies previously marked with the fluorescent dyes PE (phycoeritrin) or APC (allophycocyanin) were used as detection antibodies. The detection antibodies were diluted together in TBS to a final concentration of 1.25 ng ml⁻¹ for each antibody. For each RC, 20 μl antibody solutions were applied and incubated for 1 h at RT. After this time, the plate was washed three times with TBS and sealed with a foil.

The measurement was performed in a TIRF (Total Internal Reflection Fluorescence) microscope (Leica) with a 100-fold oil immersion objective. For this purpose, the glass bottom of the microtiter plate was generously coated with immersion oil and the plate was inserted into the automated stage of the microscope. Thereafter, one consecutive image was taken per RC at 5×5 positions in two fluorescence channels (Ex/Em=633/715 nm, 561/600 nm and 488/600 nm). The maximum laser power (100%), an exposure time of 500 ms and a gain value of 800 were chosen. The image data were then evaluated. Intensity threshold values were set for each channel at about 25% gray levels of the total intensity. In the evaluation step, the intensity threshold value was first applied for each image in each channel and images of the same position were compared with one another in both values. Only those pixels per image were counted in which, in both channels, the pixel lies at exactly the same position above the intensity threshold value of the channel. Finally, the number of pixels is averaged over all images in each RC, then the mean values of the mean pixel numbers of the replica values are determined and the standard deviation is specified.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### REFERENCES

[1] Thery, C., L. Zitvogel, and S. Amigorena, Exosomes: Composition, biogenesis and function. Nature Reviews Immunology, 2002. 2(8): p. 569-579.
[2] Wang, W. and M. T. Lotze, Good things come in small packages: exosomes, immunity and cancer. Cancer Gene Therapy, 2014. 21(4): p. 139-141.
[3] De Toro, J., et al., Emerging roles of exosomes in normal and pathological conditions: new insights for diagnosis and therapeutic applications. Frontiers in Immunology, 2015. 6.
[4] Carvalho, J. and C. Oliveira, Extracellular Vesicles—powerful markers of cancer EVolution. Frontiers in Immunology, 2015. 5.
[5] Boulanger, C. M., et al., Extracellular vesicles in coronary artery disease. Nature Reviews Cardiology, 2017. 14(5): p. 259-272.
[6] Gamez-Valero, A., et al., Urinary extracellular vesicles as source of biomarkers in kidney diseases. Frontiers in Immunology, 2015. 6.
[7] EL Andaloussi, S., et al., Extracellular vesicles: biology and emerging therapeutic opportunities. Nature Reviews Drug Discovery, 2013. 12(5): p. 348-358.
[8] Benito-Martin, A., et al., The new deal a potential role for secretec vesicles in innate immunity and tumor progression. Frontiers in Immunology, 2015. 6: p. 1-13.
[9] Erdbrugger, U. and J. Lannigan, Analytical Challenges of Extracellular Vesicle Detection: A Comparison of Different Techniques. Cytometry Part A, 2016. 89a(2): p. 123-134.
[10] Maas, S. L. N., et al., Possibilities and limitations of current technologies for quantification of biological extracellular vesicles and synthetic mimics. Journal of Controlled Release, 2015. 200: p. 87-96.
[11] Saenz-Cuesta, M., M. Mittelbrunn, and D. Otaegui, Editorial: Novel clinical applications of extracellular vesicles. Frontiers in Immunology, 2015. 6: p. 1-2.
[12] Janissen, R., L. Oberbarnscheidt, and F. Oesterhelt, Optimized straight forward procedure for covalent surface immobilization of different biomolecules for single molecule applications. Colloids and Surfaces B-Biointerfaces, 2009. 71(2): p. 200-207.

1. A method for detecting extracellular vesicles in a sample, comprising the following steps:
a) applying the sample to a substrate,
b) adding probes suitable for detection which mark the extracellular vesicles by specific binding to them; and
c) detecting the extracellular vesicles by measuring a specific signal from the probes, wherein step b) can be performed prior to step a).

2. The method according to claim 1,
wherein
prior to step a), capture molecules for the extracellular vesicles are immobilized on the substrate.

3. The method according to claim 2,
wherein
by contacting the capture molecules, the extracellular vesicles are immobilized on the substrate by binding to the capture molecules.

4. The method according to claim 1,
wherein
after the extracellular vesicles have contacted the probes, molecules and particles that are not specifically bound are removed by washing.

5. The method according to claim 1,
wherein
probes are selected which bind to the extracellular vesicles, wherein the probes are also capable of emitting a specific signal.

6. The method according to claim 2,
wherein
the extracellular vesicles contact the capture molecules and the probes simultaneously.

7. The method according to claim 2,
wherein
the extracellular vesicles contact the probes prior to contacting the capture molecules.

8. The method according to claim 1,
wherein
the sample is fixed before the probes bind to the extracellular vesicles.

9. The method according to claim 1,
wherein
the sample is treated with detergents.

10. The method according to claim 5,
wherein
a spatially resolved determination of the probe signal takes place.

11. The method according to claim 1,
wherein
the substrate comprises plastic, silicon or silicon dioxide.

12. The method according to claim 1,
wherein
the substrate has a hydrophilic surface prior to immobilizing capture molecules on the substrate.

13. The method according to claim 12,
wherein
the hydrophilic layer is selected from the group comprising or consisting of PEG, poly-lysine, dextran, and derivatives thereof.

14. The method according to claim 1,
wherein
a functionalization with amino groups occurs by bringing the substrate into contact with APTES (3-aminopropyl-trietoxy silane) or ethanolamine.

15. The method according to claim 14,
wherein
bringing the substrate into contact with APTES (3-aminopropyl-trietoxy silane) occurs in the gas phase.

16. The method according to claim 2,
wherein
the capture molecules are covalently bonded to the substrate or to a coating.

17. The method according to claim 2,
wherein
binding sites of the extracellular vesicles are epitopes and the capture molecules and probes are antibodies or parts thereof.

18. The method according to claim 1,
wherein
the probes are marked with fluorescent dyes.

19. The method according to claim 1,
wherein
detection takes place by spatially resolving fluorescence microscopy.

20. A kit for carrying out the method according to claim 1, comprising:
a substrate having a hydrophilic surface and/or an immobilized capture molecule;
a probe; and
solutions and buffers.
